(19) United States
(12) Patent Application Publication
(10) Pub. No.: US 2007/0191325 A1
(43) Pub. Date: Aug. 16, 2007

(54) Methods for altering insulin secretion

(76) Inventor: Florian Lang, Tuebingen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/591,909

(22) PCT Filed: Feb. 10, 2005
(86) PCT No.: PCT/EP05/01322

§ 371 Date: Sep. 7, 2006

(30) Foreign Application Priority Data
Mar. 8, 2004 (EP) 04005404.1

(51) Int. Cl. A61K 31/573

(52) U.S. Cl. 514/179

(57) ABSTRACT

Modulation of the activity of glucocorticoid inducible kinase SGK1 in pancreatic islet cells restores insulin release. Also disclosed are methods and compounds useful for the treatment of glucocorticoid induced diabetes mellitus type-2.

### FIELD OF THE INVENTION

A method for altering insulin secretion comprising, contacting a pancreatic islet cell expressing SGK1 with a substance that modulates SGK1 and wherein the inhibition of SGK1 involves reversal of the depolarizing effect of glucose, causing activation of voltage gated Calcium-channels and insulin release.

### BACKGROUND OF THE INVENTION

Glucocorticoid treatment induces diabetes mellitus type-2 which is readily reversible after drug withdrawal (Hoogwerf and Danese 1999; Schacke et al., 2002). In addition to peripheral insulin resistance and increased hepatic glucose production by stimulating gluconeogenesis (McMahon et al., 1988) glucocorticoids interfere with insulin secretion of pancreatic cells (Lambillotte et al., 1997; Pierluissi et al., 1986). Despite extensive studies, the molecular mechanism is still a matter of debate. The antiprogestin mifepristone (RU486), an antagonist of the nuclear glucocorticoid receptor, completely abolished Dexamethasone-induced inhibition of insulin secretion (Lambillotte et al., 1997), pointing to the involvement of glucocorticoid-dependent gene expression.

Among the glucocorticoid sensitive genes is the serum and glucocorticoid inducible kinase SGK1 (Webster et al., 1993b; Webster et al., 1993a, U.S. Pat. No. 6,326,181). SGK1 is influenced by a number of stimuli (Lang et al. 2001) such as for instance the mineral corticoids (Chen et al. 1999, Naray-Fejes-Toth et al. 1999, Shigaev et al. 2000, Brennan et al. 2000, Cowling et al. 2000).

SGK1 has been shown to be regulated through Insulin like growth factor IGF1, Insulin and through oxidative stress via a signal cascade involving phosphoinositol-3-kinase (PI3 kinase) and phosphoinositol-dependent kinase PDK1 (Kobayashi & Cohen 1999, Park et al. 1999, Kobayashi et al. 1999). The activation of SGK1 through PDK1 involves phosphorylation of Serine 422. It has furthermore been shown, that a mutation of ser 422 to aspartate (^{S422D}SGK1) results in a continuatively activated kinase (Kobayashi et al. 1999).

For the measurement of glucocorticoid inducible kinase SGK1 activity various assay systems are available. In scintillation proximity assay (Sorg et al., J. of. Biomolecular Screening, 2002, 7, 11-19) and flashplate assay the radioactive phosphorylation of a protein or peptide as substrate with γATP will be measured. In the presents of an inhibitory compound no or decreased radioactive signal is detectable. Furthermore homogeneous time-resolved fluorescence resonance energy transfer (HTR-FRET), and fluorescence polarization (FP) technologies are useful for assay methods (Sills et al., J. of Biomolecular Screening, 2002, 191-214). Other non-radioactive ELISA based assay methods use specific phospho-antibodies (AB). The phospho-AB binds only the phosphorylated substrate. This binding is detectable with a second peroxidase conjugated anti sheep antibody by chemiluminescence (Ross et al., 2002, Biochem. J., immediate publication, manuscript BJ20020786).

Earlier results showed that SGK1 is a potent stimulator of the renal epithelial Na⁺-canal (De la Rosa et al. 1999, Boehmer et al. 2000, Chen et al. 1999, Naray-Fejes-Toth et al. 1999, Lang et al. 2000, Shigaev et al. 2000, Wagner et al. 2001).

Another finding related to SGK1 was that single nucleotide polymorphism (SNP) in exon 8 with nucleotide combinations of (CC/CT) and additional polymorphism in intron 6 (CC) are associated with increased blood pressure (Busjahn et al. 2002) and from this it was concluded that SGK1 may be important to blood pressure regulation and hypertension.

Because increased activity of SGK1 correlates with renal epithelial Na⁺ channel activity which leads to hypertension through the increase of renal resorption of sodium (Lifton 1996; Staessen et al., 2003; Warnock 2001), it was conclusive that depending on the combination of allelic variants of SGK1 an increase in renal Na⁺-resorption may occur which in turn will increase the blood pressure (Busjahn et al. 2002).

Up to now insulin-secreting cells of the pancreatic islets have not been shown to express relevant amounts of SGK1 (Klingel et al. 2000) and it is generally believed that untreated islet cells do not or only to a minor extent express SGK1.

High dose Glucocorticoid treatment over an extended time period predisposes to the development of diabetes mellitus at least in part through impairment of insulin secretion. The underlying mechanism has remained elusive and targets that would allow therapeutic intervention are currently unknown. The current application defines such a new mechanism and molecular target and at the same time teaches how to identify new compounds that interfere with the fore mentioned pathomechanism with the aim to overcome diabetes mellitus.

### SUMMARY OF THE INVENTION

The current application unexpectedly demonstrates that pancreatic islet cells show a pronounced increase of SGK1 transcript levels und expression in insulin-secreting islet cells, which have been pretreated by glucocorticoids.

Glucocorticoid excess predisposes to the development of diabetes mellitus at least in part through impairment of insulin secretion and the current inventive method is for modulating the activity of SGK1 in pancreatic islet cells thereby reducing glucocorticoid induced diabetes mellitus type-2 in a subject in need of such a treatment.

The invention teaches among other aspects methods for the identification of therapeutically active compounds that are useful to restore insulin secretion by contacting a pancreatic islet cell expressing SGK1 with a substance that modulates SGK1. Thus the depolarizing effect of glucose is reversed resulting in activation of voltage gated calcium-channels and subsequent insulin release.

Modulation of SGK1 is especially useful when applied to a clinically relevant phenotype or genotype which is defined by a single nucleotide polymorphism of the SGK1 gene. Therefore the analysis of a polymorph SGK1 SNP variant in samples derived from an individual in need of treatment may be another application. Furthermore the invention delivers a method to determine the progression, regression or onset of a disease by measuring the expression of SGK1. Samples taken from the diseased individuals may furthermore allow the analysis of selected SGK1 SNP variants and their correlation with predisposition for a disease or other conditions induced for example by prolonged treatment with Glucocorticoid. Another aspect is related to screening methods for identifying new drug candidates that modulate disease related SGK1. Modulators especially useful are compounds that interfere with SGK1 function thus resulting in up-regulation of insulin secretion. Inhibitors of SGK1 are especially useful to treated subjects suffering from symptoms of diabetes mellitus type-2. Modulators of SGK1 are as well useful to treat subjects with stress induced hyperglycemia or subjects having hypoglycemia.

The drug screening method performed according to this invention has led to the discovery of SGK1 directed therapeutic compounds. Two different classes of compounds, one belonging to the class of Acylhydrazone derivatives and the other belonging to Pyridopyrimidine derivatives have been identified. Selected SGK1 inhibiting compounds in pharmaceutical compositions comprising a pharmaceutically effective carrier, excipient or diluent are useful for treatment of glucocorticoid induced diabetes mellitus type-2. It is central to this invention that the screening methods used to identify new drugs with the desired therapeutic profile are not restricted to the compounds disclosed in this application. Moreover it is evident to the expert that a one step approach or a two step approach for screening of SGK1 modulating compounds may be useful to apply. The first step of such a screening includes the identification of compounds that interfere with the SGK1 kinase activity. Various assay formats are available and a preferred assay uses the measurement of SGK1 catalysed radioactive phosphorylation of a protein or peptide as substrate together with the γATP. In the presence of an SGK1 inhibitory compound no or decreased radioactive signal is detectable. In a second step of the screening the SGK1 inhibiting compounds are tested for their potential to restore insulin secretion in glucocorticoid d treated pancreatic islet cells such as for instance INS-1 cells upon SGK1 inhibition the release of insulin is measured however measuring other read-out activities may be useful as well.

### DETAILED DESCRIPTION OF THE INVENTION

The underlying mechanism of Glucocorticoid induced diabetes has up to now remained elusive. In this invention it is shown that glucocorticoids such as Dexamethasone up-regulate transcription and expression of the serum and glucocorticoid inducible kinase SGK1 in insulin secreting cells, an effect that can be reversed by Mifepristone (RU486), an antagonist of the nuclear glucocorticoid receptor. When coexpressed in Xenopus oocytes SGK1 increases the activity of voltage-gated K⁺ channel Kv1.5. In INS-1 cells dexamethasone stimulates the transcription of Kv1.5, increases the repolarizing outward current and decreases glucose induced insulin release. The latter two effects are reversed by K⁺ channel blockers 4-AP and TEA. Dexamethasone virtually abolishes the glucose induced insulin release of islets isolated from wild type mice, an effect significantly attenuated in islets isolated from SGK1 knockout mice. In conclusion, glucocorticoids stimulate the transcription of SGK1 which in turn upregulates the activity of voltage gated K⁺ channels. The subsequent hyperpolarisation counteracts the depolarising effect of glucose and prevents the activation of voltage gated Ca²⁺ channels, Ca²⁺ entry and insulin release.

The present invention relates to the role of SGK1 and SGK1 dependent channel activity in the regulation of insulin secretion.

According to real time PCR the SGK1 transcript level is low in untreated INS-1 cells (FIG. 1A), a finding paralleling the low transcript levels reported previously for human pancreatic islets (Klingel et al., 2000). However, incubation of INS-1 cells with 100 nM dexamethasone for 2 to 23 hours increased mRNA transcript levels, an effect which was completely abrogated by the glucocorticoid receptor antagonist RU486 (FIG. 1A). Within 23 hours dexamethasone increased the cellular SGK1 transcript levels increased in mouse islets following treatment with dexamethasone (FIG. 1A). Similarly strong stimulation of SGK1 transcription by glucocorticoids was observed in other cell types (Itani et al., 2002; Rozansky et al., 2002). As apparent from Western blotting, the SGK1 protein was not detectable in untreated cells but appeared already within 2 hours and increased further within the next 23 hours exposure to dexamethasone (100 nM) (FIG. 1B). The increase in SGK1 protein abundance was fully reversed by RU486. Thus, dexamethasone stimulates the expression of SGK1 in insulin secreting cells.

As shown in FIG. 1D, coexpression of SGK1 and Kv channels in Xenopus oocytes, upregulates approximately 2-fold the activity of heterologously expressed Kv1.5 channels (FIG. 1D). Those channels have previously been shown to be expressed in INS-1 cells (Su et al., 2001) as well as rodent and human β cells (Philipson et al., 1994; Roe et al., 1996). In INS-1 cells the channels are inhibited by the K⁺ channel blocker 4-AP (Su et al., 2001). As illustrated in FIGS. 2A and 2B, treatment with dexamethasone was indeed followed by an increase in 4-AP sensitive voltage gated outward current. In untreated cells, the K⁺ channel blocker 4-AP inhibited only 10% (0.1 mM) and 28% (1 mM) of the outward current. Following a 4 hours treatment with 100 nM dexamethasone, the 4-AP sensitive current increased to 28% (0.1 mM 4-AP) and 40% (1 mM 4-AP). These data show that Kv1.5 channel activity is augmented by dexamethasone in insulin secreting cells. Glucocorticoids have been found to increase the expression of Kv 1.5 channels in heart (Takimoto and Levitan 1994), in skeletal muscle and pituitary but not in hypothalamus and lung (Levitan et al., 1996). Furthermore, dexamethasone was necessary for T3 to increase Kv1.5 mRNA levels in the rat left ventricle from adrenalectomized animals rendered hypothyroid (Nishiyama et al., 1997). Real time PCR reveals that dexamethasone (100 nM) treatment within 4 hours increases the abundance of Kv1.5 mRNA in INS cells by a factor of approx. 10. Thus, dexamethasone stimulates the expression of SGK1 which in turn increases Kv channel activity.

Additional experiments were performed to elucidate the impact of Kv channels and SGK1 on the blunting of insulin release by dexamethasone. As illustrated in FIG. 3, pretreatment of INS-1 cells with dexamethasone (100 nM) inhibited glucose-induced insulin secretion by 62%. This inhibition was reversed by Kv channel blockers, TEA and 4-AP, showing that dexamethasone mediated inhibition of insulin secretion depends on Kv channel activity.

To estimate the contribution of SGK1 to the inhibitory effect of dexamethasone on insulin secretion the effects of dexamethasone on insulin secretion in SGK1 knockout mice (sgk^{-/-}) as compared to that in wild type littermates (sgk1^{+/+}) was studied. Without dexamethasone pretreatment insulin secretion following exposure to glucose (16.7 mM), activation of adenylate cyclase (5 μM forskolin), or stimulation of protein kinase C (100 nM PMA) was not significantly different in islets isolated from sgk1^{-/-} and from sgk1^{+/+} mice (FIGS. 4 A and B, black bars). Dexamethasone treatment, however, decreased the stimulatory effect of glucose, forskolin or PMA on insulin secretion significantly more in sgk1^{+/+} islets than in sgk1^{-/-} islets. These data indicate that SGK1 participates in the downregulation of insulin secretion by dexamethasone.

In conclusion, the present experiments disclose a novel mechanism in the regulation of insulin secretion. The glucococorticoid dexamethasone enhances the transcription and expression of SGK1 in insulin secreting cells. The kinase upregulates voltage gated K⁺ channels including Kv1.5. Overexpression of Kv channels hyperpolarizes the β-cell plasma membrane thus impeding the activation of voltage gated Ca²⁺ channels. Accordingly, the kinase contributes to the inhibition of insulin release during glucocorticoid excess.

### BRIEF DESCRIPTION OF THE DRAWINGS

FIG. 1: Dexamethasone induces the expression of SGK1 in insulin secreting INS-1 cells.

INS-1 cells were treated with 100 nM dexamethasone or vehicle (DMSO) in culture for the indicated periods of time. Dexamethasone significantly induced expression of SGK1 within 2 hours. RU486 at 1 μmol/l completely inhibited the dexamethasone effect.

(A) Cellular RNA was transcribed into cDNA using reverse transcriptase M-MuLV (Roche Diagnostics GmbH, Roche Applied Science, Mannheim, Germany). SGK1 mRNA was quantified by real time PCR in a light cycler system (Roche Diagnostics GmbH, Roche Applied Science, Mannheim, Germany). The primers used were: SGK1 up: 5′-TTT TTT TTC CCA ACC CTT GC-3′; down: 5′-AAT GAA CAA AGG TTG GGG GG-3. Shown are mean±SEM of the indicated number of experiments.

(B) Whole cell lysates were subjected to 1% SDS-PAGE and plotted onto a nitrocellulose membrane (Schleicher and Schuell, Dassel, Germany). Plots were incubated with antibodies against SGK1 (New England Biolabs, Beverly, Mass., USA). Bound antibody was visualized using a second antibody coupled to horse radish peroxidase.

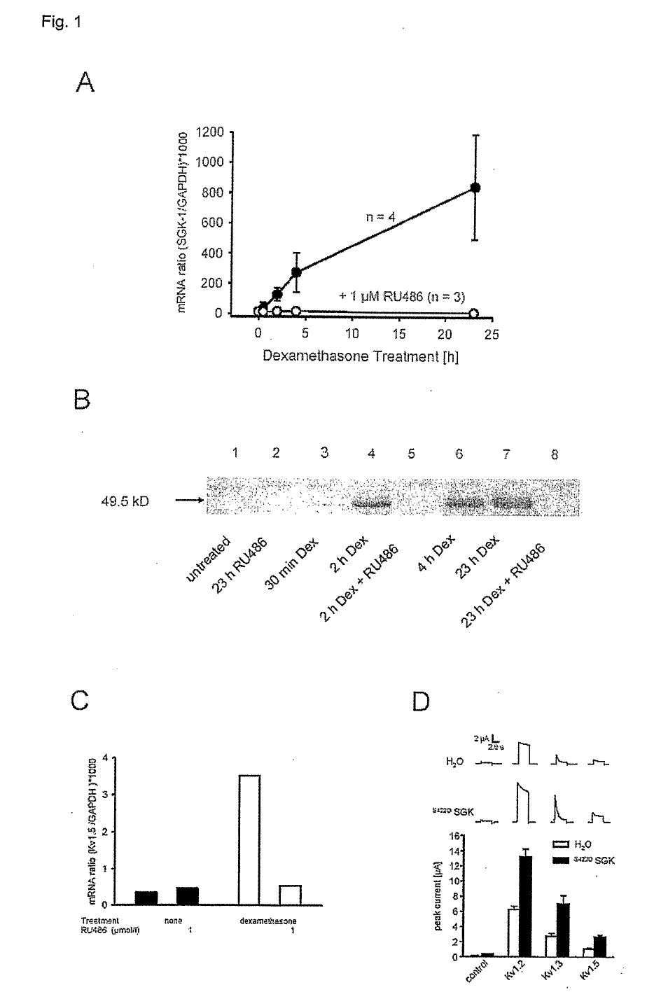

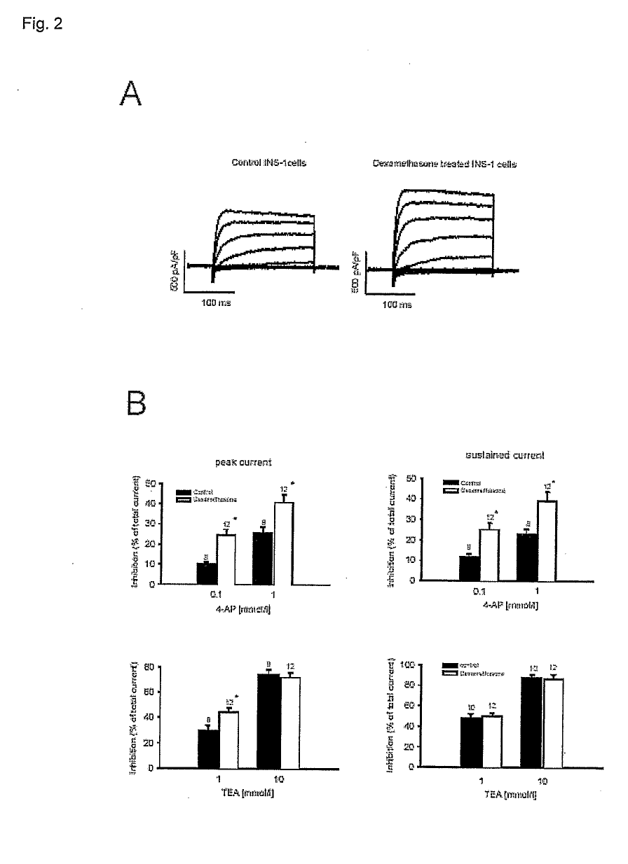

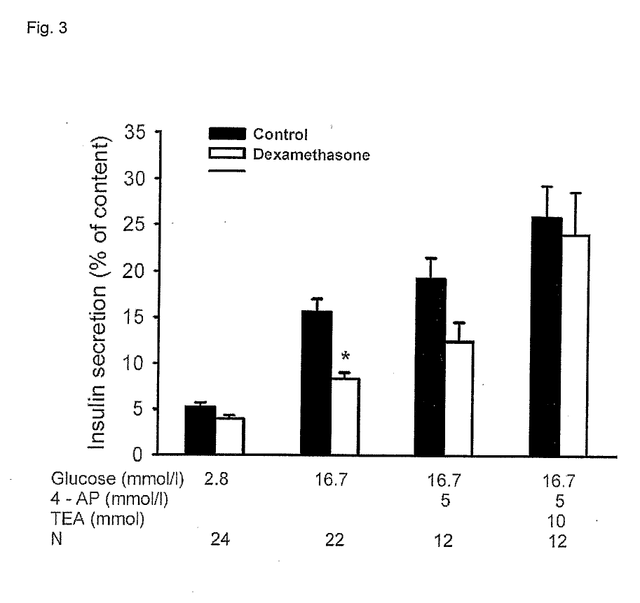

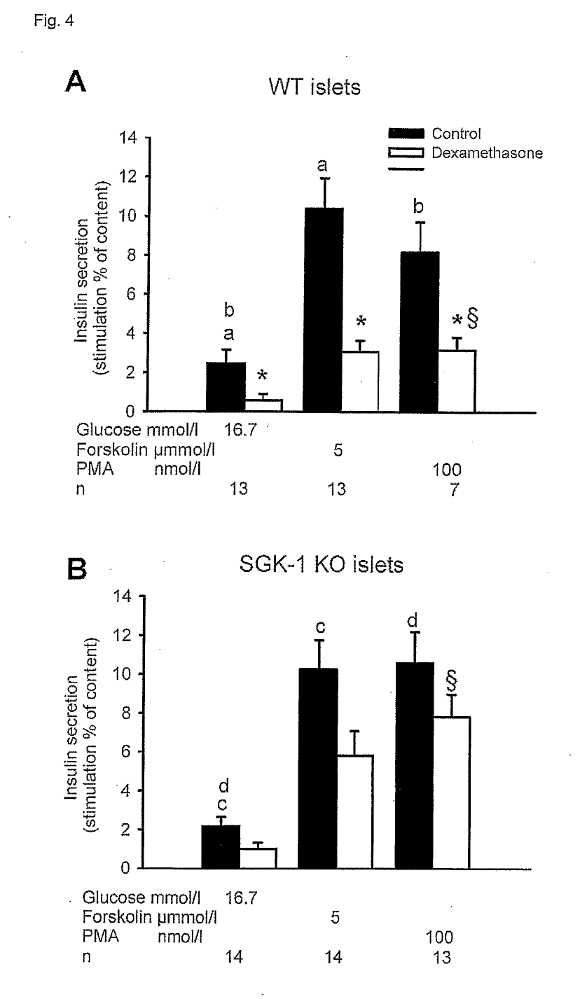

(C) Real time PCR for Kv1.5 was performed using a light cycler system (Roche Diagnostics GmbH, Roche Applied Science, Mannheim, Germany). The same RNA preparations as for the experiments described in FIG. 1A were analyzed. Shown are mean±SEM of 3 independent experiments.

(D) SGK1 and Kv channel coexpression in Xenopus oocytes increases K⁺ currents. mRNA for human SGK1 (μg/ml) and Kv 1.5 (μg/ml) was injected into oocytes and whole cell currents measured using the 2-voltage clamp method 2 days after injection. Shown are representative traces and mean±SEM.

FIG. 2: Dexamethasone augments kv channel activity in INS-1 cells.

Cells were treated prior to the experiment with 100 nm dexamethasone for 4 h. (2A) whole cell current was induced by 200 ms voltage pulses increasing by 10 mv steps from −70 mv to +50 mv. (2B) Sensitivity to 4-ap (0.1 and 1 mm) and tea (1 and 10 mm) was tested in cells before (black columns) and after dexamethasone treatment (white columns). Voltage pulses of 200 ms duration from −70 to 50 mv were applied. Shown are means±sem for the indicated number of experiments. * denotes significance (p<0.05) to current in control, non treated cells at the same inhibitor concentration. Ins-1 cells were cultured as described before (Abel et al., 1996; Asfari et al., 1992). The external patch clamp solution contained (in mmol/l): 140 NaCl, 5.6 KCl, 1.2 MgCl₂, 2.6 CaCl₂, 0.5 glucose and 10 HEPES, PH 7.4. The internal solution contained (in mmol/l): 30 KCL, 95 K⁺-gluconate, 1 MgCl₂, 1.2 NaH₂PO₄, 4.8 Na₂HPO4, 5 Na₂ATP, 1 Na₃GTP, 5 mmol/l EGTA, PH 7.2. An Epc9 Patch Clamp Amplifier (Heka Electronic, Lambrecht, Germany) Was Used For Current Measurements.

FIG. 3: Kv channel inhibition reverses dexamethasone-induced inhibition of insulin secretion of INS-1 cells.

Prior to the experiment INS-1 cells were treated in culture with dexamethasone, 100 nM, for 4 h. Cells were washed twice and preincubated in HEPES buffered salt solution containing (in mmol/l): 140 NaCl, 5.6 KCl, 1.2 MgCl₂, 2.6 CaCl₂, 0.5 glucose, 10 HEPES and 5 g/l bovine serum albumin, pH 7.4 at 37° C. Thereafter cells were incubated for 30 min at 37° C. in fresh solution containing the test substances at the appropriate concentrations. Insulin was measured by radioimmunoassay using rat insulin antiserum (Linco, Biotrend Chemikalien GmbH, Cologne, Germany), I¹²⁵-insulin (CIS Diagnostik GmbH, Dreieich, Germany) and rat insulin (Novo Nordisk, Mainz, Germany) as standard or by an Insulin Elisa kit (Mercodia, Uppsala, Sweden).

FIGS. 4 A and B: Dexamethasone did not affect secretion from islets of SGK1 knockout mice.

Isolated islets were cultured over night in RPMI 1640 containing 11 mmol/l glucose. Dexamethasone (100 ng/ml) or DMSO (control) were added 5 h before the experiment. After culture islets were preincubated for 1 h at 37° C. in incubation buffer containing (in mmol/l): 140 NaCl, 5.6 KCl, 1.2 MgCl₂, 2.6 CaCl₂, 2.8 glucose, 10 HEPES, pH 7.4 and 5 g/l bovine serum albumin (fraction V, Sigma, Deisenhofen). Thereafter, batches of 5 islets/0.5 ml were incubated for 30 min at 37° C. in the presence of test substances as indicated for each experiment. Insulin was measured using an Elisa kit (Mercodia, Uppsala, Sweden).

### ADDITIONAL METHODS AND MATERIALS

### Example 1

### Generation of Sgk1−/− Mice

A conditional targeting vector was generated from a 7-kb fragment encompassing the entire transcribed region on 12 exons (Wulff et al., 2002). The neomycin resistance cassette was flanked by two loxP sites and inserted into intron 11. Exons 4-11, which code for the sgk1 kinase domain, were “floxed” by inserting a third loxP site into intron 3. A clone with a recombination between the first and third loxP site (type I recombination) was injected into C57BL/6 blastocytes. Male chimeras were bred to C57BL/6 and 129/SvJ females. Heterozygous sgk1-deficient mice were backcrossed to 129/SvJ wild-type mice for two generations and then intercrossed to generate homozygous sgk1−/− and sgk1+/+ littermates.

### Example 2

### Cell Culture and Measurement of Insulin Secretion

INS-1 cells (kindly provided by CB Wollheim, University of Geneva, Switzerland) derived from a rat insulinoma were cultured in HEPES-buffered RPMI 1640 supplemented with 10% fetal calf serum (Biochrom, Berlin, Germany), 1 mmol/l HEPES, 1 mmol/l Na pyruvate, 10 μmol/l β-mercaptoethanol (Sigma, Munich, Germany) and antibiotics as described elsewhere (Abel et al., 1996; Asfari et al., 1992). Cells were seeded at a cell density of 2.0-2.5 10⁵ cells/ml in 24-well culture plates and cultured for 2 days prior to the experiment. Cells were washed twice with HEPES buffered salt solution containing (in mmol/l): 140 NaCl, 5.6 KCl, 1.2 MgCl₂, 2.6 CaCl₂, 0.5 glucose, 10 HEPES and 5 g/l bovine serum albumin, pH 7.4. and preincubated for 30 min at 37° C. Thereafter medium was discarded and fresh medium containing the test substances at the appropriate concentrations added. Cells were incubated for 30 min at 37° C. Incubations were stopped on ice, medium removed and frozen at −20° C. until insulin released into the supernatant was measured by radioimmunoassay using rat insulin antiserum (Linco, Biotrend Chemikalien GmbH, Cologne, Germany), I¹²⁵-insulin (CIS Diagnostik GmbH, Dreieich, Germany) and rat insulin (Novo Nordisk, Mainz, Germany) as standard or an insulin Elisa kit (Mercodia, Uppsala, Sweden). Insulin content was measured after extraction with acid ethanol (1.5 (v/v) % HCl/75% ethanol) over night at 4° C.

For isolation of islets from SGK1 KO and wild type littermates mice 3 ml of collagenase solution containing 1 mg/ml collagenase (Serva, Heidelberg, Germany) was injected into the pancreas in situ through the ductus coledochus. The entire gland was removed and digested for 10 min at 37° C. Thereafter the islets were isolated from the exocrine tissue by collecting them into fresh medium under a dissection microscope. Islets were cultured over night in RPMI 1640 containing 11 mmol/l glucose and dexamethasone (100 ng/ml) or DMSO (control). After culture islets were preincubated for 1 h at 37° C. in incubation buffer containing (in mmol/l): 140 NaCl, 5.6 KCl, 1.2 MgCl₂, 2.6 CaCl₂, 2.8 glucose, 10 HEPES, pH 7.4 and 5 g/l bovine serum albumin (fraction V, Sigma, Deisenhofen). Thereafter, batches of 5 islets/0.5 ml were incubated for 30 min at 37° C. in the presence of test substances as indicated for each experiment. Insulin was measured using an Elisa kit (Mercodia, Uppsala, Sweden).

### Example 3

### Measurement of Membrane Currents

INS-1 cells were cultured for 2-4 days on glass cover slips coated with poly-L-ornithine (10 mg/l Sigma, Munich, Germany) at appropriate cell densities (1.2×10⁶ cells/ml). The cover slips were mounted in a bath chamber on the stage of an inverted microscope (IM, Zeiss, Jena, Germany). The cells were kept at room temperature or at 34° C. as indicated for each experiment and superfused with a solution containing (in mmol/l): 140 NaCl, 5.6 KCl, 1.2 MgCl₂, 2.6 CaCl₂, 0.5 glucose and 10 HEPES, pH 7.4. The patch clamp pipettes (Clark-Medical, Reading, Great Britain) with a resistance of 4-6 MΩ were pulled using a DMZ-universal puller (Zeitz, Augsburg, Germany). They were filled with an internal solution containing (in mmol/l): 30 KCl, 95 K⁺-gluconate, 1 MgCl₂, 1.2 NaH₂PO₄, 4.8 Na₂HPO₄, 5 Na₂ATP, 1 Na₃GTP, 5 mmol/l EGTA, pH 7.2. An EPC9 patch clamp amplifier (Heka Electronic, Lambrecht, Germany) was used for current measurements. Only stable current measurements, i.e. when currents reached at least 90% of control current after removal of the respective inhibitory drug, were used for analysis.

### Example 4

### Real Time PCR

INS-1 cells were cultured in 70 cm² flasks, medium was removed and 600 μl of lysis buffer (Mini kit, Qiagen, Hilden, Germany) added. Lysed cells were scraped and the lysate collected into an Eppendorf tube. Cellular RNA was isolated using the Qiagen Mini kit and 2 μg of RNA transcribed into cDNA using Reverse Transcriptase M-MuLV (Roche Diagnostics GmbH, Roche Applied Science, Mannheim, Germany). An aliquot of cDNA, corresponding to the amount of RNA as indicated in each experiment was used for quantification of mRNA by real time PCR using a light cycler system (Roche Diagnostics GmbH, Roche Applied Science, Mannheim, Germany) with specific primers for rat Kv1.5 channel, sense: 5′-ATC TTC AAG CTC TCC CGC CAC TCC AAG GG-3′; antisense: 5′-GGG TTA TGG AAA GAG GAG TTA-3′. The primers of rat SGK1 used were: sense: 5′-TTT TTT TTC CCA ACC CTT GC-3′; antisense: 5′-MT GM CM AGG TTG GGG GG-3. Isolated mouse islets were cultured and treated with Dexamethasone as indicated. Thereafter the islets were collected and lysed in lysis buffer (Mini kit, Qiagen, Hilden, Germany) and by repeatedly sucking of the islets into an insulin syringe.

### Example 5

### Western Blotting

INS-1 cells were cultured in 70 cm² flasks without (control) or with 100 ng/ml Dexamethasone for the indicated period of time. Thereafter, culture medium was removed and cells were lysed in a solution containing 300 mM NaCl, 20 mM Tris HCl, pH 7.4, 1% (v/v) Triton X-100, 1% Sodiumdeoxycholate, 0.1% SDS, 2.5 mM EDTA, 10 μg/ml Pepstatin A, 10 μg/ml Aprotinin and 0.1 mM PMSF. Total cellular protein, 50 μg, quantified by Coomassie Blue G staining (Bradford dye assay, Biorad Laboratories GmbH, Munich, Germany) was subjected to SDS-PAGE (1%), and plotted onto a nitrocellulose membrane (Schleicher and Schuell, Dassel, Germany). Plots were incubated with antibodies against SGK1 (New England Biolabs, Beverly, Mass., USA). Bound antibody was visualized using a second antibody coupled to horse radish peroxidase.

### Example 6

### SGK1 Modulating Compounds

6.1. Compounds of the General Formula I and Pharmaceutical Useful Derivates, Salts, Solutions and Stereoisomeres Thereof Including Mixtures. 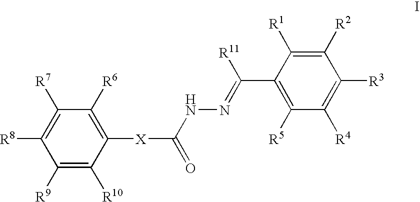
wherein
R¹, R⁵ is either H, OH, OA, OAc or Methyl,
R², R³, R⁴, R⁶, R⁷, R⁸, R⁹, R¹⁰ is either
H, OH, OA, OAc, OCF₃, Hal, NO₂, CF₃, A, CN, OSO₂CH₃, SO₂CH₃, NH₂ or COOH,
   R¹¹ H or CH₃,
   A Alkyl with 1, 2, 3 or 4 C-atoms,
   X CH₂, CH₂CH₂, OCH₂ or —CH(OH)—,
   Hal F, Cl, Br or I
   Compound According to Formula I Selected from the Following Group of Compounds:
(3-Hydroxy-phenyl)-acidic acid-(4-hydroxy-2-methoxy-benzyliden)-hydrazid,
(3-Hydroxy-phenyl)-acidic acid-[1-(4-hydroxy-2-methoxy-phenyl)-ethyliden]-hydrazid,
(3-Methoxy-phenyl)-acidic acid-(4-hydroxy-2-methoxy-benzyliden)-hydrazid.
Phenylacidic acid-(3-fluor-4-hydroxy-benzyliden)-hydrazid,
(4-Hydroxy-phenyl)-acidic acid-(4-hydroxy-2-methoxy-benzyliden)-hydrazid,
(3,4-Dichlor-phenyl)-acidic acid-(4-hydroxy-2-methoxy-benzyliden)-hydrazid,
m-Tolyl-acidic acid-(4-hydroxy-2-methoxy-benzyliden)-hydrazid,
o-Tolyl-acidic acid-(4-hydroxy-2-methoxy-benzyliden)-hydrazid,
(2-Chlor-phenyl)-acidic acid-(4-hydroxy-2-methoxy-benzyliden)-hydrazid,
(3-Chlor-phenyl)-acidic acid-(4-hydroxy-2-methoxy-benzyliden)-hydrazid,
(4-Fluor-phenyl)-acidic acid-(4-hydroxy-2-methoxy-benzyliden)-hydrazid,
(2-Chlor-4-fluor-phenyl)-acidic acid-(4-hydroxy-2-methoxy-benzyliden)-hydrazid,
(3-Fluor-phenyl)-acidic acid-(4-hydroxy-2-methoxy-benzyliden)-hydrazid,
(3-Methoxy-phenyl)-acidic acid-(4-hydroxy-benzyliden)-hydrazid, (3-Methoxy-phenyl)-acidic acid-(4-hydroxy-2,6-dimethyl-benzyliden)-hydrazid,
(3-Methoxy-phenyl)-acidic acid-(3-fluor-4-hydroxy-benzyliden)-hydrazid, (3-Methoxy-phenyl)-acidic acid-[1-(4-hydroxy-2-methoxy-phenyl)-ethyliden]-hydrazid,
(3-Methylsulfonyloxy-phenyl)-acidic acid-(4-hydroxy-2-methoxy-benzyliden)-hydrazid,
(3,5-Dihydroxy-phenyl)-acidic acid-(4-hydroxy-2-methoxy-benzyliden)-hydrazid,
(3-Fluor-phenyl)-acidic acid-(3-fluor-4-hydroxy-benzyliden)-hydrazid,
(3-Methoxy-phenyl)-acidic acid-(4-acetoxy-2-methoxy-benzyliden)-hydrazid,
(3-Trifluormethyl-phenyl)-acidic acid-(4-hydroxy-2-methoxy-benzyliden)-hydrazid,
3-(3-Methoxy-phenyl)-propionsäure-(4-hydroxy-2-methoxy-benzyliden)-hydrazid,
(3-Methoxy-phenyl)-acidic acid-(2,4-dihydroxy-benzyliden)-hydrazid,
(3-Methoxy-phenoxy)-acidic acid-(4-hydroxy-2-methoxy-benzyliden)-hydrazid,
(3-Nitro-phenyl)-acidic acid-(4-hydroxy-2-methoxy-benzyliden)-hydrazid,
(3-Methoxy-phenyl)-acidic acid-(5-chlor-2-hydroxy-benzyliden)-hydrazid,
(3-Methoxy-phenyl)-acidic acid-(2-hydroxy-5-nitro-benzyliden)-hydrazid,
2-Hydroxy-2-phenyl-acidic acid-(4-hydroxy-2-methoxy-benzyliden)-hydrazid,
(3-Methoxy-phenyl)-acidic acid-(2-ethoxy-4-hydroxy-benzyliden)-hydrazid,
(3-Brom-phenyl)-acidic acid-(4-hydroxy-2-methoxy-benzyliden)-hydrazid,
(3-Methoxy-phenyl)-acidic acid-[1-(4-hydroxy-phenyl)-ethyliden]-hydrazid,
(3,5-Difluor-phenyl)-acidic acid-(4-hydroxy-2-methoxy-benzyliden)-hydrazid,
(3-Hydroxy-phenyl)-acidic acid-(4-hydroxy-2-methyl-benzyliden)-hydrazid,
(3-Hydroxy-phenyl)-acidic acid-(2-ethoxy-4-hydroxy-benzyliden)-hydrazid,
(3-Hydroxy-phenyl)-acidic acid-(2-methoxy-4-hydroxy-6-methyl-benzyliden)-hydrazid,
(2-Fluor-phenyl)-acidic acid-(2-methoxy-4-hydroxy-benzyliden)-hydrazid
   6.2. Compounds of the General Formula II and Pharmaceutical Useful Derivates, Salts, Solutions and Stereoisomeres Thereof Including Mixtures. 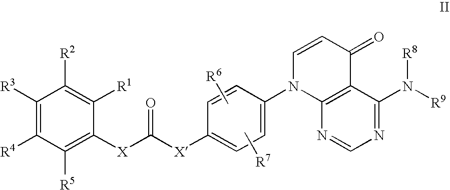
   wherein
   R¹, R², R³,
   R⁴, R⁵ is either H, A, OH, OA, Alkenyl, Alkinyl, NO₂, NH₂, NHA, NA₂, Hal, CN, COOH, COOA,
   —OHet, —O-Alkylen-Het, —O-Alkylen-NR⁸R⁹ or CONR⁸R⁹,
   two groups selected from R¹, R², R³, R⁴, R⁵ or as well —O—CH₂—CH₂—, —O—CH₂—O— or —O—CH₂—CH₂—O—,
      R⁶, R⁷ is either H, A, Hal, OH, OA or CN,
      R⁸, R⁹ is either H or A,
      Het
      Is a saturated or unsaturated heterocycle with 1 to 4 N-, O- and/or S-atoms, substituted by one or several Hal, A, OA, COOA, CN or Carbonyloxigen (═O)
      A Alkyl with 1 to 10 C-atoms, wherein 1-7H-atoms may be replaced by F and/or Chlorine,
      X, X′ is either NH or is missing
      Hal F, Cl, Br or I
      Compound According to Formula II Selected from the Following Group of Compounds:
1-[4-(4-Amino-5-oxo-5H-pyrido[2,3-d]pyrimidin-8-yl)-phenyl]-3-(2-fluor-5-trifluormethyl-phenyl)-harnstoff,
1-[4-(4-Amino-5-oxo-5H-pyrido[2,3-d]pyrimidin-8-yl)-phenyl]-3-(4-chlor-5-trifluormethyl-phenyl)-harnstoff,
1-[4-(4-Amino-5-oxo-5H-pyrido[2,3-d]pyrimidin-8-yl)-phenyl]-3-(2,4-difluor-phenyl)-harnstoff,
1-[4-(4-Amino-5-oxo-5H-pyrido[2,3-d]pyrimidin-8-yl)-phenyl]-3-(2,6-difluor-phenyl)-harnstoff,
1-[4-(4-Amino-5-oxo-5H-pyrido[2,3-d]pyrimidin-8-yl)-phenyl]-3-(3-fluor-5-trifluormethyl-phenyl)-harnstoff,
1-[4-(4-Amino-5-oxo-5H-pyrido[2,3-d]pyrimidin-8-yl)-phenyl]-3-(4-fluor-5-trifluormethyl-phenyl)-harnstoff,
1-[4-(4-Amino-5-oxo-5H-pyrido[2,3-d]pyrimidin-8-yl)-phenyl]-3-(4-methyl-5-trifluormethyl-phenyl)-harnstoff,
1-[4-(4-Amino-5-oxo-5H-pyrido[2,3-d]pyrimidin-8-yl)-phenyl]-3-(2,3,4,5,6-pentafluor-phenyl)-harnstoff,
1-[4-(4-Amino-5-oxo-5H-pyrido[2,3-d]pyrimidin-8-yl)-phenyl]-3-(2,4-dibrom-6-fluor-phenyl)-harnstoff,
1-[4-(4-Amino-5-oxo-5H-pyrido[2,3-d]pyrimidin-8-yl)-phenyl]-3-(2-fluor-6-trifluormethyl-phenyl)-harnstoff,
1-[4-(4-Amino-5-oxo-5H-pyrido[2,3-d]pyrimidin-8-yl)-phenyl]-3-(2-fluor-5-methyl-phenyl)-harnstoff,
1-[4-(4-Amino-5-oxo-5H-pyrido[2,3-d]pyrimidin-8-yl)-phenyl]-3-(2,3,4-trifluor-phenyl)-harnstoff,
1-[4-(4-Amino-5-oxo-5H-pyrido[2,3-d]pyrimidin-8-yl)-phenyl]-3-(4-brom-2,6-difluor-phenyl)-harnstoff,
1-[4-(4-Amino-5-oxo-5H-pyrido[2,3-d]pyrimidin-8-yl)-phenyl]-3-(2-fluor-3-trifluormethyl-phenyl)-harnstoff,
1-[4-(4-Amino-5-oxo-5H-pyrido[2,3-d]pyrimidin-8-yl)-phenyl]-3-[2-(1-tert.-butyloxycarbonyl-piperidin-4-yl)-phenyl]-harnstoff,
N-[4-(4-Amino-5-oxo-5H-pyrido[2,3-d]pyrimidin-8-yl)-phenyl]-2,4-dichlor-benzamid,
N-[4-(4-Amino-5-oxo-5H-pyrido[2,3-d]pyrimidin-8-yl)-phenyl]-4-chlor-5-trifluormethyl-benzamid,
N-[4-(4-Amino-5-oxo-5H-pyrido[2,3-d]pyrimidin-8-yl)-phenyl]-2-fluor-5-trifluormethyl-benzamid,
1-[4-(4-Amino-5-oxo-5H-pyrido[2,3-d]pyrimidin-8-yl)-phenyl]-3-[3-chlor-5-trifluormethyl-2-(piperidin-4-yloxy)-phenyl]-harnstoff,
1-[4-(4-Amino-5-oxo-5H-pyrido[2,3-d]pyrimidin-8-yl)-phenyl]-3-[(2-fluor-5-(2-dimethylamino-ethoxy)-phenyl]-harnstoff,
1-[4-(4-Amino-5-oxo-5H-pyrido[2,3-d]pyrimidin-8-yl)-phenyl]-3-[5-fluor-2-(piperidin-4-yloxy)-phenyl]-harnstoff,
1-[4-(4-Amino-5-oxo-5H-pyrido[2,3-d]pyrimidin-8-yl)-phenyl]-3-[4-chlor-5-trifluormethyl-2-(piperidin-4-yloxy)-phenyl]-harnstoff,
1-[4-(4-Amino-5-oxo-5H-pyrido[2,3-d]pyrimidin-8-yl)-phenyl]-3-[2-(piperidin-4-yloxy)-phenyl]-harnstoff,
1-[4-(4-Amino-5-oxo-5H-pyrido[2,3-d]pyrimidin-8-yl)-phenyl]-3-[2-fluor-5-(2-diethylamino-ethoxy)-phenyl]-harnstoff,
1-[4-(4-Amino-5-oxo-5H-pyrido[2,3-d]pyrimidin-8-yl)-phenyl]-3-[2-fluor-5-[2-(piperidin-1-yl)-ethoxy]-phenyl]-harnstoff,
1-[4-(4-Amino-5-oxo-5H-pyrido[2,3-d]pyrimidin-8-yl)-phenyl]-3-[4-fluor-2-(2-dimethylamino-ethoxy)-phenyl]-harnstoff,
1-[4-(4-Amino-5-oxo-5H-pyrido[2,3-d]pyrimidin-8-yl)-phenyl]-3-[4-fluor-2-(2-diethylamino-ethoxy)-phenyl]-harnstoff,
1-[4-(4-Amino-5-oxo-5H-pyrido[2,3-d]pyrimidin-8-yl)-phenyl]-3-[3-chlor-4-[2-(morpholin-4-yl)-ethoxy]-phenyl]-harnstoff,
1-[4-(4-Amino-5-oxo-5H-pyrido[2,3-d]pyrimidin-8-yl)-phenyl]-3-[4-fluor-2-[2-(morpholin-4-yl)-ethoxy]-phenyl]-harnstoff,
1-[4-(4-Amino-5-oxo-5H-pyrido[2,3-d]pyrimidin-8-yl)-phenyl]-3-[3-chlor-4-(2-dimethylamino-ethoxy)-phenyl]-harnstoff,
1-[4-(4-Amino-5-oxo-5H-pyrido[2,3-d]pyrimidin-8-yl)-phenyl]-3-[3-chlor-4-(2-diethylamino-ethoxy)-phenyl]-harnstoff,
1-[4-(4-Amino-5-oxo-5H-pyrido[2,3-d]pyrimidin-8-yl)-phenyl]-3-[4-chlor-2-(2-dimethylamino-ethoxy)-phenyl]-harnstoff,
1-[4-(4-Amino-5-oxo-5H-pyrido[2,3-d]pyrimidin-8-yl)-phenyl]-3-[2-chlor-5-(2-diethylamino-ethoxy)-phenyl]-harnstoff,
sowie ihre pharmazeutisch verwendbaren Derivate, Solvate, Salze, Tautomere und Stereoisomere, einschlieβlich deren Mischungen in alien Verhältnissen.

### Example 8

### SGK1 Nucleotide Polymorphism

The nucleotide sequence defining intron 6 of facultative hypertensive patients is . . . aattacattgCgcaacccag . . . , whereas the nucleotide sequence representing a healthy population is . . . aattacattTgcaacccag . . . . Both sequences are available through accession number GI 2463200 Position 2071.

The exon 8 sequences of facultative hypertensive patients are either homozygotic . . . tactgaCttcggact . . . or . . . tactgaTttcggact . . . or heterozygotic .tactgaCttcggact . . . and . . . tactgaTttcggact. The sequences are available through accession number NM_{—}005627.2, Position 777.

A homozygotic individual with a TT nucleotide combination is protected even if simultaneously a CC single nucleotide polymorphism is presented in intron 6.

### Example 9

### Statistics

Data are presented as mean±SEM. ANOVA for multiple groups and Student's t-tests were used for statistical analysis. p values<0.05 were accepted to indicate statistical significance.

### REFERENCES

Böhmer, C., Wagner, C. A., Beck, S., Moschen, I., Meizig, J., Werner, A., Lin, J.-T., Lang, F., Wehner, F. The Shrinkage-activated Na⁺ Conductance of Rat Hepatocytes and its Possible Correlation to rENaC. Cell Phys Biochem. 2000; 10:187-194.
Brenan F E, Fuller P J. Rapid upregulation of serum and glucocorticoid-regulated kinase (sgk) gene expression by corticosteroids in vivo. Mol Cell Endocrinol. 2000; 30; 166:129-36.
Busjahn A, Aydin A, Uhlmann R, Feng Y, Luft F C, Lang F. Serum- and glucocorticoid-regulated kinase (SGK1) gene and blood pressure. Hypertension 40(3): 256-260, 2002
Chen S Y, Bhargava A, Mastroberardino L, Meijer O C, Wang J, Buse P, Firestone G L, Verrey F, Pearce D: Epithelial sodium channel regulated by aldosterone-induced protein sgk. Proc Natl Acad Sci USA 1999; 96:2514-2519.
Cowling R T, Birnboim H C. Expression of serum- and glucocorticoid-regulated kinase (sgk) mRNA is up-regulated by GM-CSF and other proinflammatory mediators in human granulocytes. J Leukoc Biol. 2000; 67:240-248.
De la Rosa D A, Zhang P, Naray-Fejes-Toth A, Fejes-Toth G, Canessa C M: The serum and glucocorticoid kinase sgk increases the abundance of epithelial sodium channels in the plasma membrane of Xenopus oocytes. J Biol Chem 1999; 274:37834-37839.
Hoogwerf B, Danese R D: Drug selection and the management of corticosteroid-related diabetes mellitus. Rheum Dis Clin North Am 1999; 25:489-505.
Klingel K, Wärntges S, Bock J, Wagner C A, Sauter M, Waldegger S., Kandolf R, Lang F. Expression of the cell volume regulated kinase h-sgk in pancreatic tissue. Am J Physiol (Gastroint. Liver-Physiol.) 2000; 279:G998-G1002.
Kobayashi T, Cohen P: Activation of serum- and glucocorticoid-regulated protein kinase by agonists that activate phosphatidylinositide 3-kinase is mediated by 3-phosphoinositide-dependent protein kinase-1 (PDK1) and PDK2. Biochem J 1999; 339:319-328.
Kobayashi T. Deak M, Morrice N, Cohen P. Characterization of the structure and regulation of two novel isoforms of serum- and glucocorticoid-induced protein kinase. Biochem. J. 1999; 344:189-197.
Lambillotte C, Gilon P, Henquin J C: Direct glucocorticoid inhibition of insulin secretion. An in vitro study of dexamethasone effects in mouse islets. J Clin Invest 1997; 99:414-423.
Lang F, Cohen P. Regulation and physiological roles of serum- and glucocorticoid-induced protein kinase isoforms. Science STKE. 2001 Nov. 13; 2001 (108):RE17.
Lang F, Klingel K, Wagner C A, Stegen C, Warntges S, Friedrich B, Lanzendörfer M, Melzig J, Moschen I, Steuer S, Waldegger S, Sauter M, Paulmichl M, Gerke V, Risler T, Gamba G, Capasso G, Kandolf R, Hebert S C, Massry S G, Bröer S: Deranged transcriptional regulation of cell volume sensitive kinase hSGK in diabetic nephropathy. Proc Natl Acad Sci USA 2000; 97:8157-8162.
Lifton R P. Molecular genetics of human blood pressure variation. Science 1996; 272:676-680.
McMahon M, Gerich J, Rizza R: Effects of glucocorticoids on carbohydrate metabolism. Diabetes Metab Rev 1988; 4:17-30.
Naray-Fejes-Tóth A, Canessa C, Cleaveland E S, Aldrich G, Fejes-Tóth G: sgk is an aldosterone-induced kinase in the renal collecting duct. Effects on epithelial Na⁺ channels. J Biol Chem 1999; 274:16973-16978.
Park J, Leong M L, Buse P, Maiyar A C, Firestone G L, Hemmings B A: Serum and glucocorticoid-inducible kinase (SGK) is a target of the PI 3-kinase-stimulated signaling pathway. EMBO J. 1999; 18:3024-3033.
Pierluissi J, Navas F O, Ashcroft S J: Effect of adrenal steroids on insulin release from cultured rat islets of Langerhans. Diabetologia 1986; 29:119-121.
Schacke H, Docke W D, Asadullah K: Mechanisms involved in the side effects of glucocorticoids. Pharmacol Ther 2002; 96:23-43.
Shigaev A, Asher C, Latter H, Garty H, Reuveny E: Regulation of sgk by aldosterone and its effects on the epithelial Na(+) channel. Am J Physiol 2000; 278:F613-F619.
Staessen J A, Wang J, Bianchi G, Birkenhager W H. Essential hypertension. Lancet. 2003; 361:1629-1641.
Wagner C A, Ott M, Klingel K, Beck S, Melzig J, Friedrich B, Wild N K, Bröer S, Moschen I, Albers A, Waldegger S, Tumler B, Egan E, Geibel J P, Kandolf R, Lang F. Effects of serine/threonine kinase SGK1 on the epithelial Na⁺ channel (EnaC) and CFTR. Cell Physiol Biol 2001; 11:209-218.
Warnock D G. Liddle syndrome: genetics and mechanisms of Na+ channel defects. Am J Med Sci. 2001; 322:302-307.
Webster M K, Goya L, Firestone G L: Immediate-early transcriptional regulation and rapid mRNA turnover of a putative serine/threonine protein kinase. J Biol Chem 1993a; 268:11482-11485.
Webster M K, Goya L, Ge Y, Maiyar A C, Firestone G L: Characterization of sgk, a novel member of the serine/threonine protein kinase gene family which is transcriptionally induced by glucocorticoids and serum. Mol Cell Biol 1993b; 13:2031-2040.
   6.2. Compounds of the General Formula II and Pharmaceutical Useful Derivates, Salts, Solutions and Stereoisomeres Thereof Including Mixtures. 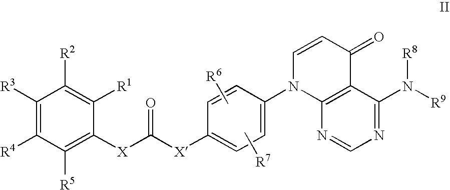
   wherein
   R¹, R², R³,
   R⁴, R⁵ is either H, A, OH, OA, Alkenyl, Alkinyl, NO₂, NH₂, NHA, NA₂, Hal, CN, COOH, COOA,
   —OHet, —O-Alkylen-Het, —O-Alkylen-NR⁸R⁹ or CONR⁸R⁹,
   two groups selected from R¹, R², R³, R⁴, R⁵ or as well —O—CH₂—CH₂—, —O—CH₂—O— or —O—CH₂—CH₂—O—,
      R⁶, R⁷ is either H, A, Hal, OH, OA or CN,
      R⁸, R⁹ is either H or A,
      Het
      Is a saturated or unsaturated heterocycle with 1 to 4 N—, O— and/or S-atoms, substituted by one or several Hal, A, OA, COOA, CN or Carbonyloxigen (═O)
      A Alkyl with 1 to 10 C-atoms, wherein 1-7H-atoms may be replaced by F and/or Chlorine,
      X, X′ is either NH or is missing
      Hal F, Cl, Br or I
      Compound According to Formula II Selected from the Following Group of Compounds:
1-[4-(4-Amino-5-oxo-5H-pyrido[2,3-d]pyrimidin-8-yl)-phenyl]-3-(2-fluor-5-trifluormethyl-phenyl)-harnstoff,

1. A method for altering insulin secretion comprising, contacting a pancreatic islet cell expressing SGK1 with a substance that modulates SGK1.

2. A method according to claim 1, wherein the expressed SGK1 comprises a selected SNP variant.

3. A method of claim 1, wherein the modulator of SGK1 is an inhibitor.

4. A method of claim 1, wherein the modulator is an activator of SGK1.

5. A method of claim 1, wherein the inhibition of SGK1 comprises reversal of the depolarizing effect of glucose, activation of voltage gated Ca-channels and insulin release.

6. A method according to claim 5, wherein the polymorph SGK1 SNP variant is diagnosed before inhibition.

7. A method according to claim 1, characterized by the up-regulation of insulin secretion

8. The method of claim 1 wherein the treated subject suffers from symptoms of diabetes mellitus type-2.

9. A method for reducing glucocorticoid induced diabetes mellitus type-2 in a subject in need of such a treatment by modulating the activity of SGK1 in pancreatic islet cells.

10. The method of claim 1, wherein the treated subject has stress induced hyperglycemia.

11. The method of claim 1, wherein the treated subject has hypoglycemia.

12. A method for determining the progression, regression or onset of a disease by measuring the expression of SGK1, comprising taking a sample from the diseased individual.

13. A method according to claim 12, wherein the SGK1 comprises a selected SNP variant.

14. A Pharmaceutical composition comprising an SGK1 inhibiting agent together with a pharmaceutically effective carrier, excipient or diluent.

15. Use of SGK1 inhibitors selected from the listed compounds having the general formula I or II for the manufacture of a medicament for the treatment of disorders caused by impaired insulin secretion.
